(19) United States
(12) Patent Application Publication
(10) Pub. No.: US 2009/0177008 A1
(43) Pub. Date: Jul. 9, 2009

(54) NOVEL PROCESS FOR SYNTHESIS OF ITOPRIDE AND ITS NOVEL INTERMEDIATE N-(4-HYDROXYBENZYL)- 3,4-DIMETHOXYBENZAMIDE

(75) Inventors: Bakulesh Mafatlal Khamar, Ahmedabad (IN); Indravadan Ambalal Modi, Ahmedabad (IN); Jayaraman Venkatraman, Ahmedabad (IN); Ponnaiah Ravi, Ahmedabad (IN); Divakara Somayajulu Nori, Ahmedabad (IN); Madhusudana Rao Gajula, Ahmedabad (IN); Gurusamy Renugadevi, Ahmedabad (IN); Kunjaru N. Shashikala, Ahmedabad (IN); Santosh M. Rudramuni, Ahmedabad (IN)

Correspondence Address: DARBY & DARBY P.C., P.O. BOX 770, Church Street Station, New York, NY, 10008-0770, US

(73) Assignee: CADILA PHARMACEUTICALS LTD., Ahmedabad (IN)

(21) Appl. No.: 12/159,291

(22) PCT Filed: Dec. 28, 2006
(86) PCT No.: PCT/IB2006/003784

§ 371 Date: Sep. 19, 2008

(30) Foreign Application Priority Data
Dec. 28, 2005 (IN) 1629/MUM/2005

(51) Int. Cl. C07C 231/10; C07C 233/60

(52) U.S. Cl. 564/176

(57) ABSTRACT

The present invention relates to a novel and improved process for the preparation of N-[4-[2-(dimethylamino)ethoxy]benzyl]-3,4-dimethoxybenzamide—known as Itopride, via a novel intermediate N-(4˜hydroxybenzyl)-3,4-dimethoxybenzamide.

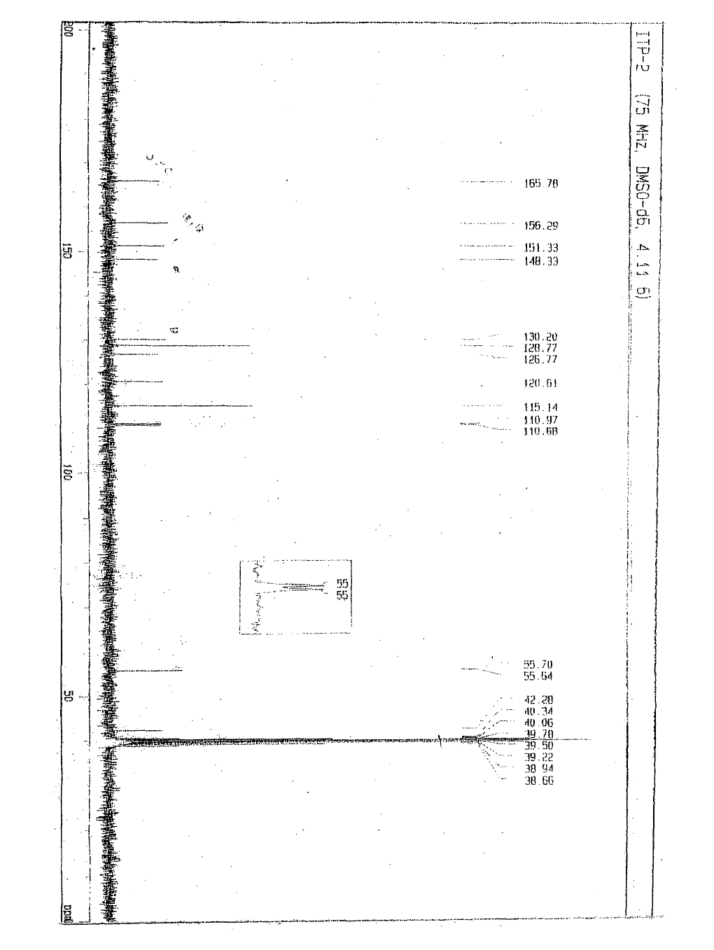

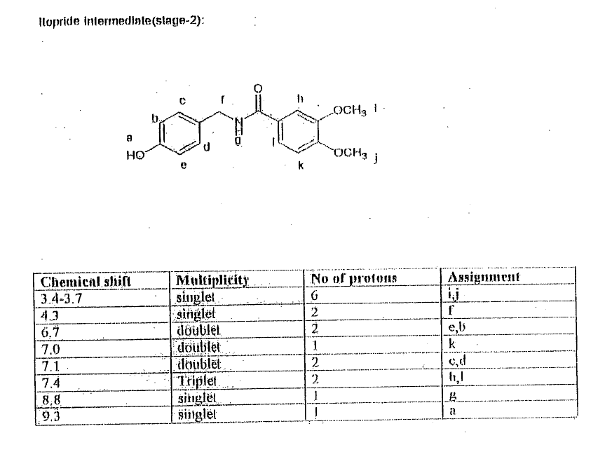

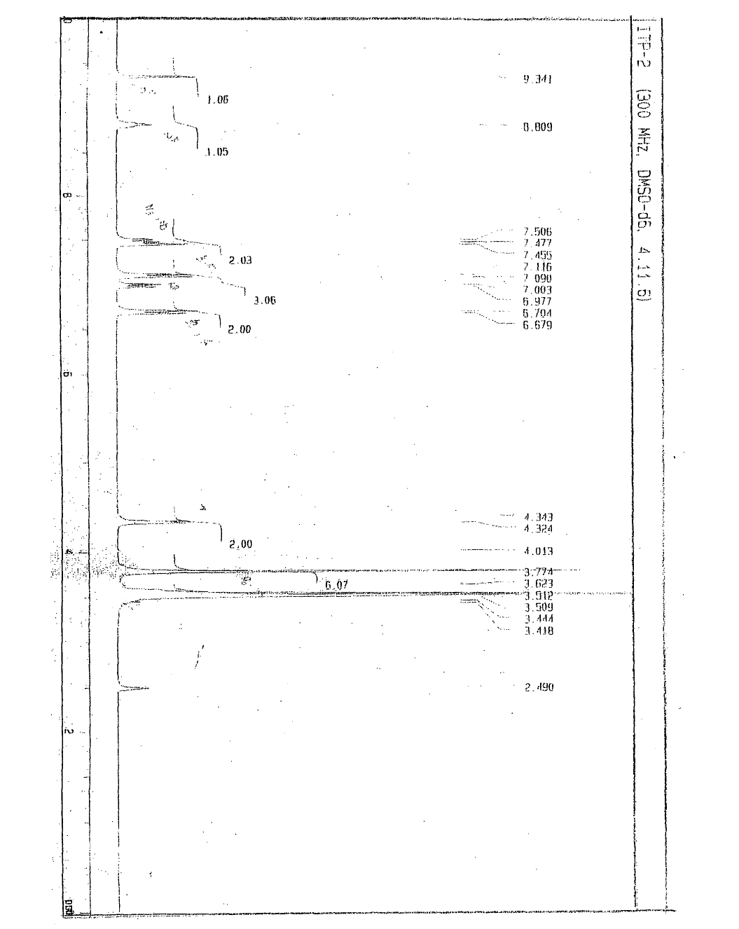

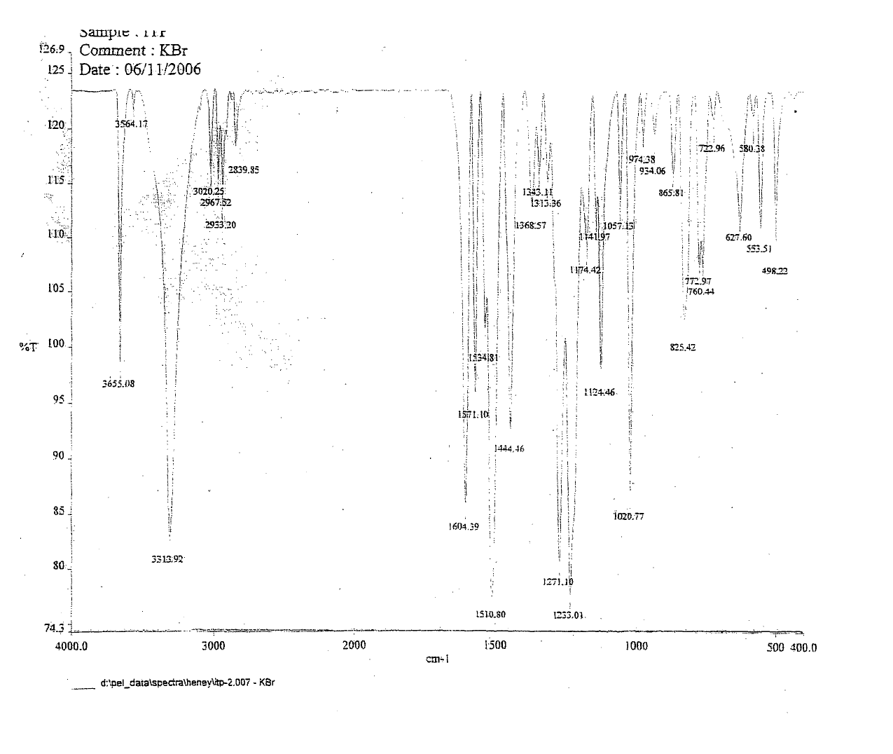

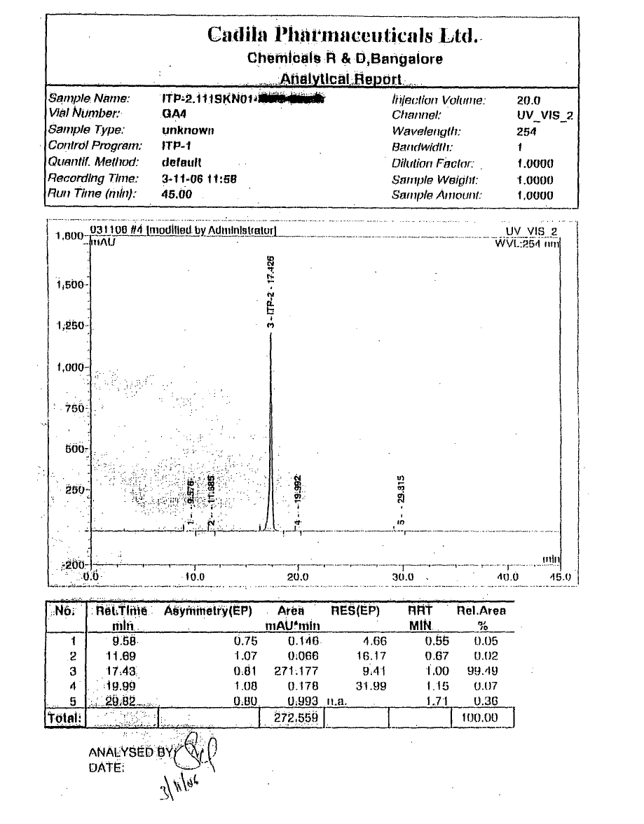

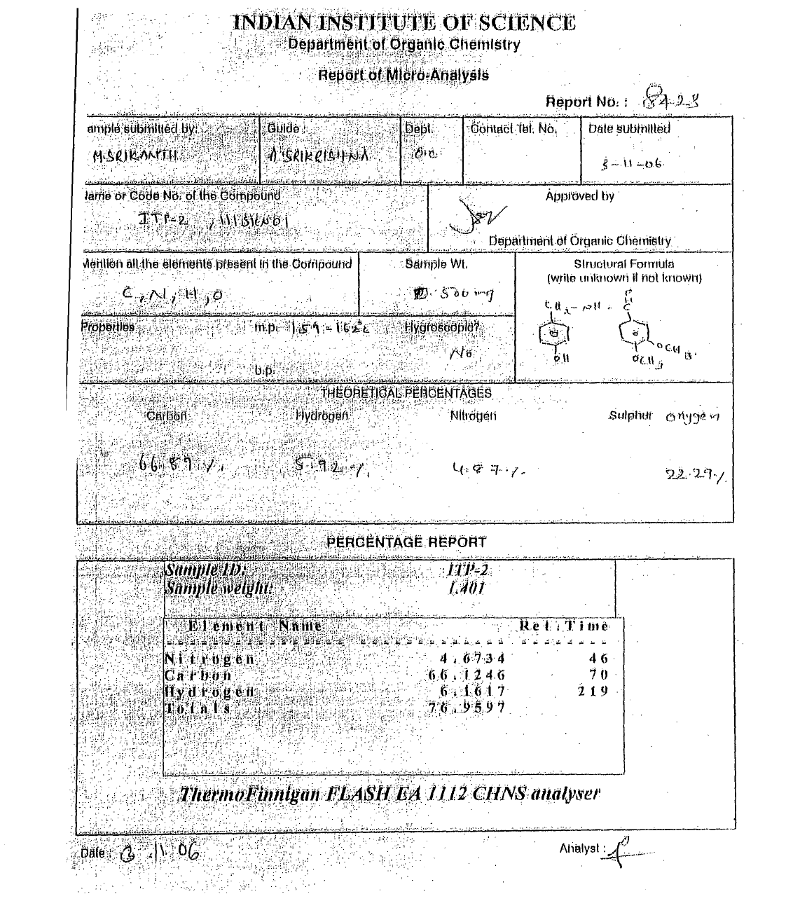

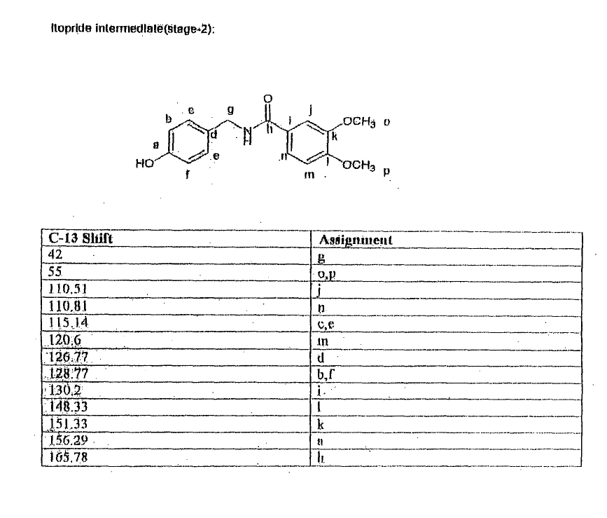

### FIELD OF THE INVENTION

The present invention relates to a process for the preparation of N-[4-[2-(dimethylamino)ethoxy]benzyl]-3,4-dimethoxybenzamide. [herein referred to as “Itopride”], via a novel intermediate N-(4-hydroxybenzyl)-3,4-dimethoxybenzamide.

### BACKGROUND OF THE INVENTION

N-[4-[2-(dimethylamino)ethoxy]benzyl]-3,4-dimethoxybenzamide (Itopride) is a prokinetic agent represented by formula-1, which is used as hydrochloride.

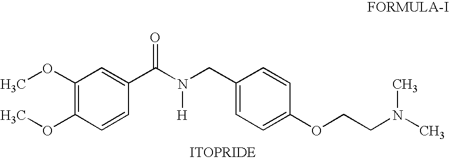

U.S. Pat. No. 4,983,633 [Equivalent to EP306827, JP01066153 A2, JP05037982 B4, JP01079144 A2, JP01085960 A2, JP01093568 A2 CA1335101 A1, AU606988 B2, AU8821862 A1] describes the preparation of Itopride hydrochloride as follows:

4-hydroxybenzaldehyde is reacted with 2-dimethylaminoethyl chloride to give 4-[2-(dimethylamino)ethoxy]benzaldehyde, which is reacted with hydroxylamine hydrochloride to give 4-[2-(dimethylamino)ethoxy]benzaldoxime, which is reduced to 4-[2-(dimethylamino)ethoxy]benzyl amine, reacted with 3,4-dimethoxybenzoyl chloride to give itopride, which is converted to its hydrochloride salt using ethanolic hydrogen chloride.

4-[2-(dimethylamino)ethoxy]benzaldehyde is prepared alternatively by reacting 4-(2-haloethoxy)benzaldehyde with dimethyl amine.

Du, Wenshuang; Pan, Li; and Cheng Maosheng in Chinese journal Shenyang Yaoke Daxue Xuebao (2003), 20(4), 260-261, 265 describe the synthesis of Itopride wherein Itopride is synthesized by etherifying 4-hydroxybenzaldehyde with N,N-dimethyl-2-chloroethylamine in the presence of K₂CO₃ at 80° C. for 2 hours, condensing with hydroxylamine HCl in ethanol under reflux for 1 hour, hydrogenating in the presence of Raney Ni at 50° C. under pressure to obtain 4-(2-dimethylaminoethoxy)benzenemethanamine (I); oxidizing 3,4-dimethoxybenzaldehyde with 20% KMnO₄ soln. at room temperature for 10 min, chlorinating with SOCl₂, and amidating with (I) in diisopropyl ether for 1 hour.

As described above, 4-[2-(Dimethylamino)ethoxy]benzyl amine is an intermediate in the preparation of itopride. The synthesis of this compound and/or its intermediates are are disclosed in JP2004231527A2, JP2004231526A2 and DE10235312.

JP 2004231527 A2 describes the synthesis of 4-(2-dimethylamino)ethoxybenzonitrile, wherein 2-(Dimethylamino)ethanol (I) is added to a mixture of PhMe, 1,3-dimethyl-2-imidazolidinone, and NaH, heated at 90° C. for 1 hour to give Na salt of —(I), which is treated with 4-chlorobenzonitrile at 110° C. for 2 hours to give ≧90% 4-(2-dimethylamino)ethoxybenzonitrile.

JP 2004231526 A2 describes the synthesis of 4-(2-dimethylamino)ethoxybenzonitrile, wherein 2-(dimethylamino)ethanol sodium salt in toluene is reacted with 4-chlorobenzonitrile at 110° C. for 2 hours resulting in yield of 90%.

DE 10235312 A1 describes the synthesis of 4-[2-(dimethylamino)ethoxy]benzylamine by the catalytic hydrogenation of 4-[2-(dimethylamino)ethoxy]benzonitrile prepared by the etherification of 4-chlorobenzonitrile with 2-(dimethylamino)ethanol in the presence of Group IA (e.g., sodium methoxide) or IIA alkoxides, in the presence of hydrogen and Raney nickel or Raney cobalt hydrogenation catalysts.

All the prior art approaches use 4-[2-(Dimethylamino)ethoxy]benzyl amine as an intermediate for the synthesis of Itopride wherein dialkylamino ethyl group is introduced in the early stage of synthesis resulting in increased consumption of 2-dimethylaminoethanol/2-dimethylamino ethyl chloride, a strong base is used for etherification step.

It is a long standing need of the industry to develop commercially viable process for the preparation of Itopride by alternate routes which result in lower consumption of 2-dimethylaminoethanol/2-dimethylaminoethyl chloride.

### SUMMARY OF THE INVENTION

The present invention is directed to improved and cost effective preparation of Itopride on commercial scale.

Yet another object of the invention is to provide a process for the preparation of Itopride via a novel intermediate-N-(4-hydroxybenzyl)-3,4-dimethoxybenzamide.

Yet another object of the invention is to provide a process for preparation of N-(4-hydroxybenzyl)-3,4-dimethoxybenzamide.

Yet another object of the present invention is to provide a process for preparation of itopride with relatively low usage of dimethylaminoethyl chloride.

Yet another object of the invention is to provide a process of preparing itopride using a phase transfer catalyst and biphasic system which may be solid- liquid or biphasic solvent system wherein one phase is water and other phase is water immiscible solvent, in the O-alkylation step.

### DETAILED DESCRIPTION OF THE INVENTION

In accordance with the present invention, N-(4-hydroxybenzyl)-3,4-dimethoxybenzamide and Itopride—a compound of formula-I are synthesized by a process comprising steps:
(a) Reacting 4-(aminomethyl)phenol with 3,4-dimethoxy benzoic acid derivatives such as 3,4-dimethoxy benzoyl halide, preferably 3,4-dimethoxy benzoyl chloride in a solvent in the presence of a base to give N-(4-hydroxybenzyl) 3,4-dimethoxybenzamide,
(b) Reacting N-(4-hydroxybenzyl) 3,4-dimethoxybenzamide with a compound of formula II

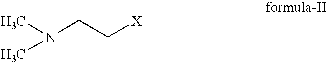

wherein X is a leaving group such as Cl, Br, I OTs, OMs, OAc OBz, preferably Cl, wherein the reaction is earned in a solvent in the presence of a base. Optionally the reaction is earned out in a solvent in the presence of a phase transfer catalyst and a base that is insoluble in the solvent. In another embodiment the reaction is carried out in a solvent in the presence of a phase transfer catalyst and a base in a biphasic system, comprising water and a water immiscible organic solvent to give itopride.

In step-(a),
the base is selected from alkali metal or alkaline earth metal carbonates and bicarbonates; tertiary amines, such as triethyl amine, tripropyl amine, dialkyl anilines, Hunig bases such as diisopropyl ethyl amine, DBU, DBN, N-alkyl morpholines and N-alkyl pyrrolidines and N-alkyl piperidines and their like, pyridine bases such as 2,4lutidine, 2,6-lutidine, s-collidine, the preferred base being triethylamine;
the solvent is selected from aromatic hydrocarbons or mixtures thereof, aliphatic hydrocarbons or mixtures thereof, nitriles or mixtures thereof, chlorinated solvents or mixtures thereof, the preferred solvent being dichloromethane.
the reaction is earned out at 5-150° C. preferably at 5 to 50° C.

In step-(b),
the solvent for the reaction is selected from N,N dialkyl amides such as N,N-dimethyl formamide, N,N-dimethyl acetamide, tetramethyl urea, N,N′-dimethyl propyleneurea, HMPT, or mixture thereof;
aromatic hydrocarbon solvents such as benzene, toluene, Xylene, ethylbenzene or mixture thereof;
alicyclic solvents such as cyclohexane, cyclopentane and their like or mixture thereof;
ketone group of solvents such as acetone, MIBK, MEK, or mixtures thereof;
ether group of solvents such as THF, Dioxane, Monoglyme, dibutyl ether, MTBE and their like or mixture thereof; the preferred being THF for the method not using PTC.

O-alkylation is earned out using a base in a solvent. Optionally the reaction may be carried out in a solvent, a phase transfer catalyst and a base that is insoluble in the solvent. In yet another embodiment the reaction may be carried out in the presence of phase transfer catalyst and a base in a biphasic solvent system in which one solvent is water and the other solvent is water immiscible organic solvent. The preferred organic solvent in biphasic solvent system is toluene. The progress of the reaction is monitored by TLC/HPLC.

Phase transfer catalyst used for O-alkylation is selected from a group of
quaternary ammonium salts of general formula N(R1R2R3R4)X wherein R1, R2, R3, and R4 is C1 to C13 alkyl or aralkyl group, cycloalkyl, aryl, or heterocyclyl. X is a monovalent anion. The preferred catalyst being triethyl benzyl ammonium chloride, or tetrabutyl ammonium bromide, or tetrabutyl ammonium hydrogen sulfate;
Quat. Phosphonium salts;
PEG ethers.

The base in step-(b) is selected from alkali metal hydroxides, alkali metal and alkaline earth metal carbonates and bicarbonates, tertiary amines such as triethyl amine, tripropyl amine, dialkyl anilines, Hunig bases such as diisopropyl ethyl amine, DBU, DBN, N-alkyl morpholines and N-Alkyl pyrrolidines and N-alkyl piperidines and their like; pyridine bases such as 2,4 lutidine, 2,6-lutidine, s-collidine; the preferred base is potassium carbonate for process not using PTC, whereas the preferred base is potassium hydroxide for process using PTC and a biphasic solvent system.

Itopride formed in step (b) is converted to its hydrochloride salt using HCl gas in a non-aqueous solvent or aqueous HCl in water miscible solvent.

For preparing hydrochloride salt using HCl gas and non-aqueous solvent, the non-aqueous solvent is selected from dichloromethane, ether, ethyl acetate, acetone, MIBK, toluene, lower alcohols such as methanol, ethanol, propanol, isopropanol, butanol preferably isopropyl alcohol.

In the preparation of hydrochloride salt using aqueous HQ, water miscible solvents such as lower alcohols such as ethanol, methanol, propanol, isopropyl alcohol; ketones such as MIBK, acetone are used. In a preferred embodiment, itopride in isopropyl alcohol, is reacted at −10 to 50° C. with HCl gas dissolved in isopropyl alcohol to give hydrochloride salt which is separated, washed with a solvent and dried in vacuum.

The invention is illustrated with non-limiting examples as follows:

### Example-1

### Preparation of P_Hydroxy Benzylamine

Methanol (2.0 L) is charged in an autoclave, p-hydroxybenzaldehyde (250 gms; 2.049M) is added followed by 25 gms (50 gms wet) of Raney Nickel, and aqueous ammonia (25%) (800 ml; 11.7647M). The hydrogenator is evacuated and flushed with nitrogen, a few times. The autoclave is initially pressurized to 3 Kg/cm² with hydrogen and then maintained at 5 Kg/cm² hydrogen pressure for 15-20 hours at 25-28° C. The reaction is monitored by TLC and continued till the starting material is less than 2%. After releasing hydrogen pressure, the reaction mixture is further heated for 30 minutes at about 40° C. The catalyst is filtered and washed with methanol, followed by purging of the solution with nitrogen for about one hour till the evolution of ammonia ceases. The solvent is distilled off at 40-45° C. to ¼ of the total volume under vacuum, cooled to 0° C. and stirred for two hours. The solid is filtered and the cake is washed with 2×250 ml of water, followed by washing with 2×250 ml of toluene. The material is dried at 70-75° C. till the moisture content is < 1%. The product analysis is as follows.

Product weight: 210-215 gms.

Purity by HPLC: 95-98%

Yield: 80-85%

Melting range: 115-119° C.

### Example-2

### Preparation of P_Hydroxy Benzylamine

Methanolic ammonia (13-15%) (700 ml) is charged into an autoclave, p-hydroxybenzaldehyde (100 gm; 0.82 moles) is charged followed by Raney Nickel (15 gms; 30 gms wet). The hydrogenator is evacuated and flushed with nitrogen, a few times. The autoclave is pressurized to 5 Kg/cm² with hydrogen and maintained at 5 Kg/cm² for 5-6 hours at 25-28° C. The reaction is monitored on HPLC and the reaction is continued till the starting material is less than 2%. Hydrogen pressure is released and the catalyst is filtered and washed with methanol. Nitrogen gas is purged in the solution for one hour till the evolution of ammonia gas ceases. The solvent is distilled off up to ⅓rd of the total volume under vacuum at 40-45° C. The reaction mass is cooled to 0° C. and stirred for two hours. The solid is filtered and washed with 2×100 ml of water, followed by 100 ml of methanol. The material is dried in oven at 50-55° C. under vacuum till the moisture content is < 1%.

Product weight: 80-85 gms.

Purity by HPLC: 95-98%

Yield: 80-85%

### Example-3

### Preparation of 3,4-Dimethoxy Benzoyl Chloride

Toluene (312 ml) is charged in a 1 L 4 neck RBF fitted with magnetic stirrer thermowel, water condenser and calcium chloride guard tube. 3,4-dimethoxy benzoic acid (78 gms; 0.4286 moles) is charged followed by N,N-dimethylformamide (3 ml). The reaction mass is heated with stirring to 40° C. Thionyl chloride, 38 ml (61.18 gms, 05143 moles) is added at 40° C. over a period of 30 minutes. The reaction mixture is heated to 50-55° C. and stir for 2 hours. The reaction is monitored by TLC. After the reaction is complete, the solvent is distilled off completely. The product is directly taken for next stage considering the yield as 98%.

### Example-4

### Preparation of N-(4-Hydroxy Benzyl)-3,4-Dimethoxy Benzamide

Dichloromethane (750 ml) is charged into a 1 L 4 neck RBF equipped with mechanical stirrer, thermowel, addition funnel and calcium chloride guard tube, p-hydroxybenzylamine (50 gms; 0.4065 moles) is charged, followed by triethyl amine (56.6 ml; 0.4065 moles). The flask is cooled to 15° C. 3,4-dimethoxybenzoyl chloride (81.5 gms; 0.4065 moles) dissolved in 100 ml of dichloromethane is added over a period of 30-45 minutes, maintaining the temperature of the reaction between 15-20° C. The reaction mixture is stirred for 30-45 minutes at 15-20° C. The progress of the reaction is monitored on HPLC and the reaction is continued till the starting material is <2%. Dichloromethane is distilled off under vacuum (at 40-45° C.). The residue is triturated with 500 ml of water and stirred for 2 hours.

The solid is filtered and washed with 3×100 ml of water. The material is dried in oven at 70-75° C. for 10-15 hours till moisture content is <3%.

Product weight: 110-112 gms.

Purity by HPLC: 94-96%

Yield: 95-97%

### Example-5

### Preparation of N,N-Dimethyl Amino Ethyl Chloride

Water (50 ml) is charged into 500 ml capacity 3 neck RBF fitted with mechanical stirrer. N,N-dimethylaminoethyl chloride hydrochloride (100 gms (0.6944 moles) is charged under stirring. The reaction mass is cooled to 5-10° C. A chilled solution of sodium hydroxide solution [36 g (0.9027 mole) in 72 ml of water] is added at 10° C., and further stirred for 15 minutes. pH of the solution is found to be around 9-10. Sodium chloride (65 gm) is added to the reaction mass, stirred and the organic layer is separated used immediately.

Product weight: 62 gms.

Purity by GC: 98-99%

Yield: 85%

### Example-6

### Preparation of Itopride

THF (400 ml) is charged in 1 L capacity multi-neck RBF equipped with mechanical stirrer, thermowel, condenser mounted on an oil bath. N-(4-hydroxy benzyl)-3,4-dimethoxy benzamide (50 gms; 0.1742 M) is charged with stirring. Anhydrous K₂CO₃ (60 gms) is charged with stirring, followed by 18.7 gms of dimethylaminoethyl chloride. The reaction mass is heated to 60-65° C. and stirred for 4 hours. 2-Dimethylaminoethyl chloride (4.7 gms) is added and the stirring is continued for 10-12 hours. The progress of the reaction is monitored by TLC. Addition of fresh 2-dimethylaminoethyl chloride is continued till TLC shows less than 3% of starting material. The reaction mass is cooled to 35-40° C., and filtered and washed with 2×50 ml THF. The solvent is distilled off under vacuum and reaction mass is dumped into 300 ml water. The pH is adjusted to 1-2 using cone. HCl (˜20 ml). The mass is stirred for 15 minutes. Ethyl acetate (200 ml) is added and stirred for 10 minutes. The layers are separated. The aqueous layer is cooled to 5-10° C. and the product is precipitated by adding 400 ml of 10% potassium carbonate solution till alkaline. (pH˜8-9). The mass is stirred for 2 hours, filtered, and the cake is washed with 3×100 ml water. The product is dried in an oven at 70-75° C. till moisture content is <1%.

Product weight: 45-49 gms.

Purity by HPLC: 98-99%

Yield: 77-79%

Melting range: 105-112° C.

### Example-7

### Preparation of Itopride

Toluene (300 ml) is charged in a 1 L multi-neck RBF equipped with mechanical stirrer, thermowel, condenser and calcium chloride guard tube mounted on an oil bath. N-(4-hydroxybenzyl)-3,4-dimethoxy benzamide (50 gms; 0.1742 moles) is charged. 19.5 gm of potassium hydroxide in 10 ml water is charged followed by 3 gms of TBAB and 24.3 gms of dimethylaminoethyl chloride under stirring. The reaction mixture is heated to 95-100° C. and stirred at the same temperature for 3 hours. The progress of the reaction is monitored by HPLC, and the stirring is continued till starting material i.e. N-(4-hydroxybenzyl)-3,4-dimethoxy benzamide is less than 2%. Water (100 ml) is charged and layers are separated at 70-75° C. The organic layer is washed two times with water. The organic layer is separated and charged back into a RB flask. The organic layer is stirred for 2 hours, the product is filtered and the product is washed with 50 ml of toluene. The material is dried in oven at 70-75° till the LOD is <1%.

Product weight: 51 gms.

Purity by HPLC: 96-98%

Yield: 81.6%

### Example-8

### Preparation of Itopride Hydrochloride

IPA (240 ml) is charged into 500 ml 3 neck RBF equipped with mechanical stirring, water condenser, calcium chloride guard tube and thermowel mounted on an oil bath. Itopride (40 gms) is charged. The reaction mass is heated to dissolve the material. Activated charcoal (4 gms) is added and the reaction mass is refluxed for 30 minutes. The hot solution (˜40 to 50° C.) is filtered over celite bed. The filtrate is transferred into other 500 ml 4 neck RBF with mechanical stirring. The pH is adjusted to 1-2 by adding IPA-HCl (˜25 ml) maintaining the temperature at 25-30° C., and stirred for 30 minutes at 25-30° C. The solid is filtered and washed with 2×25 ml of IP A. The material is dried in oven at 70-80° C. for 6-8 hours till loss on drying is less than 1%. The material (˜38-40 gms) is transferred to other 500 ml RBF and charged 120 ml of acetonitrile. The reaction mass is stirred at reflux temperature for 2-3 hours. The solid is filtered in hot condition (at 50-60° C.) and washed with 50 ml of acetonitrile. The material is air dried for 2-3 hours and then dried in oven at 70-80° C. till loss on drying is less than 1%. The yield and analysis is as follows.

Product weight: 35-36 gms.

Purity by HPLC: 99.4-99.7%

Yield: 79-81%

Melting range: 191-195° C.

1. A process of preparing itopride comprising the steps of:
(a) Reacting a solution of 4-(aminomethyl)phenol with 3,4-dimethoxy benzoic acid derivatives such as 3,4-dimethoxy benzoyl halide-, preferably 3,4-dimethoxy benzoyl chloride in a solvent with trimethyl amine to give N-(4-hydroxybenzyl) 3,4-dimethoxybenzamide;
(b) Reacting N-(4-hydroxybenzyl) 3,4-dimethoxybenzamide with a compound of formula II 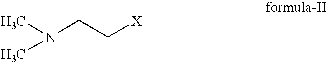
wherein X is a leaving group such as Cl, Br, I OTs, OMs, OAc OBz, preferably Cl,
wherein,
the reaction is carried out in THF as solvent in the presence of sodium carbonate or potassium carbonate;
or
in a solvent in the presence of a phase transfer catalyst and a base that is insoluble in the solvent;
or
in the presence of a phase transfer catalyst and a base in a biphasic system comprising water and a water immiscible organic solvent.

2. (canceled)

3. The process for preparing itopride as claimed in claim 1 wherein the solvent used for this reaction in step (a), is selected from aromatic hydrocarbons or mixtures thereof; aliphatic hydrocarbons or mixtures thereof; nitriles or mixtures thereof; chlorinated solvents or mixtures thereof; the preferred solvent being dichloromethane.

4. The process for preparing itopride as described in claim 1 wherein the temperature of the reaction in step (a), is carried out at 5-150° C., preferably at 5 to 50° C.

5. (canceled)

6. The process for preparing itopride as described in claim 1 wherein a solvent for the method using PTC is toluene.

7-9. (canceled)

10. A compound of formula [chemical structure]
